(19) United States
(12) Patent Application Publication
(10) Pub. No.: US 2021/0088509 A1
(43) Pub. Date: Mar. 25, 2021

(54) STABILIZED VESICLE-FUNCTIONALIZED MICROPARTICLES FOR CHEMICAL SEPARATIONS AND RAPID FORMATION OF POLYMER FRITS IN SILICA CAPILLARIES USING SPATIALLY-DEFINED THERMAL POLYMERIZATION

(71) Applicant: ARIZONA BOARD OF REGENTS ON BEHALF OF THE UNIVERSITY OF ARIZONA, Tucson, AZ (US)

(72) Inventors: Craig A. Aspinwall, Tucson, AZ (US); Jinyan Wang, Tucson, AZ (US); Kendall Sandy, Tucson, AZ (US); Steven Scott Saavedra, Tucson, AZ (US); Christopher Baker, Knoxville, TN (US); Elyssia S. Gallagher, Waco, TX (US); Boying Liang, Tucson, AZ (US)

(21) Appl. No.: 17/111,037

(22) Filed: Dec. 3, 2020

Related U.S. Application Data
(63) Continuation-in-part of application No. 15/751,125, filed on Feb. 7, 2018, now abandoned.
(60) Provisional application No. 62/203,134, filed on Aug. 10, 2015.
(60) Provisional application No. 62/203,202, filed on Aug. 10, 2015.

(51) Int. Cl. G01N 33/543

(52) U.S. Cl. CPC G01N 33/5432

(57) ABSTRACT

Surface-modified silica microparticles that are functionalized with stabilized phospholipid vesicles are described herein. These stabilized vesicles can be functionalized with either transmembrane receptors or membrane associated receptors and used for affinity pull-down assays or other chromatographic separation modalities to provide affinity capture/concentration of low abundance ligands in complex mixtures with minimal sample preparation. Further described are methods and apparatus for forming polymer frits in a fused silica capillary. The capillary containing a monomer solution is placed between one or more heat sources connected to each other via a jig and operatively coupled to a temperature controller. The polymer frits are synthesized via thermal polymerization of the monomer solution using the heat sources, which allows for placement of the polymer frits at a spatially-defined location in the capillary.

### CROSS REFERENCE

This application is a continuation-in-part and claims benefit to U.S. patent application Ser. No. 15/751,125, filed Feb. 7, 2018, which is a 371 of PCT/US16/46203, filed Aug. 9, 2016, which claims benefit to U.S. Provisional Patent Application No. 62/203,134, filed Aug. 10, 2015, and U.S. Provisional Patent Application No. 62/203,202, filed Aug. 10, 2015, the specification(s) of which is/are incorporated herein in their entirety by reference.

### GOVERNMENT SUPPORT

This invention was made with government support under Grant Nos. R01 GM095763 and R01 EB007047 awarded by National Institutes of Health. The government has certain rights in the invention.

### FIELD OF THE INVENTION

The present invention relates to bioassay platforms, in particular, to pull-down assay platforms using silica core-polymerized phospholipid vesicle shell particles for peptide/protein ligand screening.

The present invention further relates to the formation of polymer frits, in particular, to a rapid and high precision method of forming polymer frits for proteomic applications that rely on capillary LC-Mass Spectrometry. The present invention provides better control and spatial precision than other common photopolymerization approaches.

### BACKGROUND OF THE INVENTION

Many physiological or pathological events involve the molecular recognition and binding between a peptide/protein ligand and a specific target on the cell membrane. For example, bacterial infection usually starts when a bacterial pathogen or protein toxins released by the bacteria bind with glycolipids on the cell membrane. Another example is the binding between extracellular peptide ligands and transmembrane protein receptors. Specific ligand-receptor binding events trigger corresponding cellular responses, such as enzyme activity and gene expression. Nowadays, screening of molecules that bind to targets on cell membranes is an important process in drug discovery.

Cell-based functional assays are commonly used methods for ligand screening, where a specific downstream response in a signaling pathway (e.g. calcium flux) is monitored upon ligand binding. However, complicated signaling events in cells can interfere with this assay. Binding assays are also used, such as those based on labeled ligands (e.g. radio-labels) or surface plasmon resonance (SPR) with the SPR assays being label-free. Although these methods are useful, none of them can identify an unknown ligand from a ligand mixture. Hence, there is a need for rapid and highly specific assay platforms for identifying novel ligand-receptor interactions while minimizing crosstalk and non-specific binding.

The present invention features a novel pull-down assay platform for simple and effective identification of peptide/protein ligands that bind to membrane targets. The platform technology presented herein addresses a key need for biomedical and clinical analysis—the ability to use natural binding events for ligand quantitation. The present invention relies upon interactions existing in nature rather than complex and time-consuming synthesis and selection of antibodies.

Packed bed columns are used in both high-performance liquid chromatography (HPLC) and capillary electrochromatography (CEC) to achieve high-efficiency separations for a variety of applications. In liquid chromatography, packed beds are advantageous compared to open tubular columns because they provide a higher stationary phase capacity and separation efficiency, which leads to improved resolution between analytes. Packed beds allow for the use of the various particle sizes, porosities, and stationary phases that have been developed for HPLC. For many applications, capillary liquid chromatography (cLC) is superior to conventional HPLC with smaller column diameters allowing for increased sensitivity and the use of smaller volumes of analytes and mobile phase. Small cLC columns offer compatible flow rates needed for mass spectrometry and thus are used in many omics applications. However, capillary columns necessitate smaller dead volumes compared to traditional HPLC columns.

On-column fabrication of frits allows for packed cLC capillaries with minimal dead volume. Frits are needed to retain the packing material; however, reliable frit fabrication is considered one of the most difficult processes in fabricating packed capillaries. Requirements for frits include mechanical robustness to endure high packing pressures and permeability for use in chromatography.

The primary on-column capillary frit materials include silica particles and polymers. Various methods are employed to fabricate these on-column frits. The earliest method used involves sintering silica particles to crosslink the silica between the particles and the capillary wall. This method has inherent disadvantages including alteration of the stationary phase, band broadening, fragility of capillary, and difficulty in controlling pore size. Another method involves heating larger porous silica particles within a capillary. Methods for heating include using a Ni—Cr ribbon or wire powered by a DC power source, soldering gun, or a fiber optic splicer. These methods do not allow for precise control of the temperature, thus the porosity of the frits is not reproducible. Other heating methods have also been developed, such as the use of a stainless steel tube to directly apply heat when fabricating frits and using a muffle furnace to apply radial heat to silica particles at the end of capillaries. Although the stainless steel tube heating method is simple, often undesired air bubbles are introduced within the frit after the structure of the silica particles is destroyed. In both methods, the frit chemistry is limited to the silica particle surface.

One main advantage of polymer frit formation is the ability to tune the frit pore size based on the monomer composition. Either UV or thermal free radical initiation is used to polymerize monomer solutions. UV polymerization allows for control of frit length and position, with polymerization only occurring where the polyimide sheathing has been removed since the stabilizing polyimide capillary coating is not UV-transparent; however, this introduces a fragile window to the capillary. Thermal polymerization is advantageous because the capillary sheathing is maintained.

When constructing a packed bed within a capillary, two frits are necessary within the capillary: a retaining frit to pack particles against and a second retaining frit to hold the particles within the packed bed. Both sintering particles and current thermal polymerization methods for polymer frits are limited to creating frits at the end of capillaries. Therefore, a method is needed to create a second retaining frit with precise spatial location at the end of packed beds.

The present invention features a new approach for rapid synthesis of frits with spatial precision. This method has advantages including a simple preparation process, rapid polymerization times (<2 min), ease of frit placement, and retention of capillary polyimide sheathing. A comparison of the new thermal polymerization method against an established UV polymerization method is described herein.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

Multiple disease states are caused by dysregulation of biochemical pathways through ligand-receptor interactions. The screening of peptide or protein ligands that bind to targets on cell membrane (e.g. glycolipids, transmembrane protein receptors) is an important process in drug discovery. Discovery of ligands that target transmembrane proteins is limited to platforms that support protein function. Existing methods mostly rely on ligand labeling (e.g. radio-labels), the complex development of antibodies or can be interfered by complicated cellular signaling events (functional assays).

The subject disclosure features an assay platform for selecting ligands that bind to a specific receptor. Such ligands may include drug candidates, hormones, neurotransmitters, or other molecules. In some embodiments, the assay platform may comprise one or more microparticles, a plurality of lipid vesicles each comprising a spherical lipid bilayer and bonded to a surface of each microparticle, and one or more target receptors specific to the ligands. For example, the vesicles may be covalently or non-covalently bonded to the surface of microparticles. Preferably, the receptors are embedded in the lipid bilayer of the vesicle. The assay platform can be mixed into a solution comprising one or more known or unknown ligands such that a ligand selectively binds to the target receptor to form a ligand-bound assay platform, enabling identification of all ligands from the mixture that bind selectively to the receptor. The ligand-bound assay platform is removed from the solution, and can be detected via an analytical instrument. When the ligand is detected, the class of ligand is identified, and structural analysis is performed to identify the specific structure of the ligand. For example, the unknown ligand can be identified if it binds to the receptors since the identity of the receptors is known and the receptors are specific to the ligand.

Another embodiment of the subject disclosure is a method for quantifying a known ligand. The method may comprise providing any embodiment of the assay platform described herein, mixing the assay platform into a solution comprising the ligand such that the ligand binds to the target receptors to form a ligand-bound assay platform, removing the ligand-bound assay platform from the solution, and utilizing an analytical instrument to quantify the specific ligand bound to receptor of the assay platform.

A further embodiment of the subject disclosure features a method of preparing an assay platform for selecting a ligand. The method may comprise providing a plurality of microparticles, depositing modifying molecules on a surface of each microparticle to form a surface-modified microparticle, mixing a plurality of lipid monomers with one or more target receptors specific to the ligand to yield a monomer-receptor mixture, forming the monomer-receptor mixture into a plurality of lipid vesicles, polymerizing the lipid vesicles such that the receptors are embedded in a lipid bilayer of the vesicle, and depositing the vesicles on the surface of the surface-modified microparticle to form the assay platform.

According to one embodiment, a target receptor is incorporated into phospholipid vesicles ranging from 100-600 nm composed of an NH₂-functionalized lipid and a polymerizable lipid. A non-limiting example of the polymerizable lipid is 1,2-bis[10-(2′,4′-hexadieoyloxy)decanoyl]-sn-glycero-2-phosphocholine (bis-sorbPC). Polymerization of bis-sorbPC formed crosslinking structures within the lamellar region of the vesicles, greatly enhancing vesicle stability and enabling detection using a range of analytical methods, many of which are incompatible with unstabilized phospholipid vesicles. The polymerized, receptor- and NH₂-functionalized vesicles are covalently immobilized on sulfonate-modified silica microspheres, making novel silica core-vesicle shell particles. The core-shell particles can be incubated with a ligand mixture. The bound ligand can be separated from the ligand mixture by centrifugation of the particles. Finally, the bound ligand can be identified by directly exposing the particles to MALDI-MS analysis. Without wishing to limit the invention to any theory or mechanism, it is believed that polymerization can provide the lipid vesicles with enough stability to survive MS detection conditions. For instance, Cholera toxin binding unit (CTB) was successfully detected using ganglioside GM1 (CTB's membrane receptor) functionalized core-shell particles. This novel platform may be utilized for the discovery of unknown ligand/receptor pairs with minimal sample preparation. None of the presently known prior references or work has these unique inventive technical features of the present invention.

The key advance in this work is the application of stabilized lipid membranes to prepare the vesicle functionalized microparticles. Stabilization of the lipid membrane provides the key enabling step that preserves the integrity of the vesicle, and associated receptor/binding element, for a sufficient time frame to be practically useful. Vesicle stabilization can be achieved using a number of approaches, though two primary approaches are used herein. First, reactive lipid monomers that form covalent lipid polymer networks, e.g. sorbyl and dienoyl functionalized lipids, can be utilized. These lipids directly polymerize under suitable initiation conditions to form both linear and crosslinked polymer networks with varying degrees of fluidity to support receptor function. In a second approach, natural or synthetic lipids, which do not directly undergo polymerization, can be utilized to form stabilized membranes via the introduction of a secondary polymer scaffold. This polymer scaffold is prepared within the lipid bilayer, with the net result of increasing the non-covalent interactions between the lipid and the scaffold to increase vesicle stability. While other approaches have been utilized to attach non-stabilized vesicles to particle surfaces, the integration of stabilized vesicles described herein significantly enhances the functional utility and lifetime of the vesicle functionalized microparticle and the combination of reeptor functionalized, stabilized vesicle coated microparticles, enable new analytical measurements.

Another embodiment of the present invention features the formation of polymer frits. In capillary liquid chromatography (cLC), on-column porous frits are used to retain packed-bed materials. Common methods for frit synthesis include sintering silica particles and formation of polymer frits. Polymer frit synthesis most commonly uses UV-initiated polymerization, which necessitates a fragile window in the capillary sheathing. As described herein, the subject disclosure features an approach to rapid, spatially distinct frit synthesis that employs a facile yet robust thermal polymerization using a simple temperature controlled heating apparatus, enabling reproducible formation of polymer frits within a 100 μm i.d. fused-silica capillary without removal of the protective polyimide sheath.

Frits were synthesized in 3-(trimethoxysilyl)propyl methacrylate modified capillaries via free radical thermal polymerization using a monomer solution of 2,2-azobisisobutyronitrile (AIBN), glycidyl methacrylate (GMA), ethylene glycol dimethacrylate (EGDMA), and decanol. Frit length and stability were investigated as a function of polymerization time and temperature. Thermal initiated frits exhibited comparable stability to UV-polymerized frits. A packed capillary with two thermal initiated frits remained intact during cLC experiments and allowed for reproducible reverse-phase separations of aliphatic amines. This approach offers short polymerization times of <2 min compared to ≥1 h for UV polymerization. Without wishing to limit the invention to any theory or mechanism, this approach provides control of frit length and frit placement, and improved capillary stability via retention of the polyimide capillary sheathing. It is a cost-effective and robust alternative to UV-polymerization, and allows packed-capillary columns to be readily tuned for experimental application. None of the presently known prior references or work has these unique inventive technical features of the present invention.

### BRIEF DESCRIPTION OF THE DRAWINGS

The patent application or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A shows a non-limiting example of some targets on a cell membrane for peptide/protein ligand screening.

FIG. 1B shows a schematic of the silica core-polymerized phospholipid vesicle shell particle according to an embodiment of the present invention.

FIG. 2 shows a schematic of the novel pull-down assay process according to an embodiment of the present invention.

FIG. 3 shows a non-limiting example of mass spectra of silica core-polybis-sorbPC/NH₂/GM1 vesicle shell particles (top), and silica core-polybis-sorbPC/NH₂ vesicle shell particles (bottom), after incubating with CTB and after washing.

FIG. 4 shows a non-limiting example of a reaction scheme for the formation of receptor and NH₂-functionalized, polymerized phospholipid vesicles.

FIG. 5 shows non-limiting examples of polymerizable lipid monomers.

FIG. 6 depicts sulfonate-modification of silica microsphere surface and immobilization of phospholipid vesicles to the modified surfaces according to an embodiment of the present invention.

FIG. 7 shows exemplary fluorescence images of bare and sulfonate-modified silica microspheres after incubating with fluorescein and amino-fluorescein.

FIG. 8 shows zeta potential measurements of silica microspheres at different modification steps (n=3) according to an embodiment of the present invention.

FIG. 9 shows non-limiting examples of fluorescence intensity measurements of sulfonate-modified silica microspheres with 1,2-Dioleoyl-sn-glycero-3-phosphocholine (DOPC)/NH₂ and polybis-sorbPC/NH₂ vesicle coatings, without and with MeOH rinse, and stained with FM1-43 (measured by flow cytometry, n>5000).

FIG. 10A shows a schematic of silica core-shell particles with immobilized cell membrane vesicles containing membrane proteins used for high throughput ligand screening.

FIG. 10B shows NAN-190 fluorescence of silica core-shell particles with CHO-K1 vesicles containing overexpressed 5-HT1A receptors measured using flow cytometry in accordance with the embodiment shown in FIG. 10A.

FIGS. 11A-11B shows an exemplary reaction scheme for thermal frit polymerization within a fused silica capillary. FIG. 11A shows silanol on the capillary wall reacting with TMSPM to provide a methacrylate functionality on the capillary surface. FIG. 11B shows GMA (monomer), EGDMA (crosslinker), AIBN (thermal initiator) and decanol (not pictured) introduced into the capillary.

FIGS. 12A-12B show an exemplary setup for thermal polymerization using soldering irons, such as WELLER soldering irons, wherein WELLER is a registered trademark of Apex Tool Group, LLC. FIG. 12A depicts two soldering irons with 3 mm tips that are connected to variable alternating current (VAC) transformers for temperature control, such as VARIAC VAC transformers, wherein VARIAC is a registered trademark owned by Instrument Service & Equipment, Inc. A custom-built hollow aluminum alignment jig was used to stabilize the 3 mm tips of the soldering irons during polymerization. FIG. 12B shows VARIAC VAC transformers allowing for temperature control at 3 mm tips with linear regression y=3.24x−21.4 R²=0.994.

FIG. 13 is a characterization of polymer frit length (mm) versus time (s) at four different polymerization temperatures; n=3. Error bars are standard deviations.

FIGS. 14A-14B shows deviation pressures for 8 mm frits. FIG. 14A shows a determination of frit deviation pressure by deviation in linearity of equilibration pressure versus flow rate indicated by vertical line. Solid linear regression: y=48.3x−400 R²=0.99, dashed linear regression: y=88.5x−886, R²=0.99. FIG. 14B is a comparison of frit deviation pressures for 8 mm frits polymerized using labeled conditions. Error bars are standard deviations; n=3.

FIG. 15 shows a chromatogram of FITC labeled aliphatic amines using C₁₈ packed capillary with thermal initiated frits. Zonal chromatography of 125 nM FITC-labeled n-hexylamine (A) and n-octylamine (B) in ethanol with UV detection. Mobile phase: 45/55 ACN/H₂O, 0.1% TFA (v/v). Range: 0.005, A: 240 nm, 2.0 μL min⁻¹.

FIG. 16 shows an 8 cm long thermally initiated polymer frit synthesized at 92° C. for 75 s. Inset is an SEM image of frit.

FIG. 17 shows a schematic of a setup used for cLC reverse phase separation using packed capillary. A) A section of 4.5″ 250 μm i.d. PEEK tubing was connected to the outlet of the pump and connected to a 3.5″ long 100 μm i.d. capillary using a Microtight PEEK connector. B) Injection valve connected to 4″ long 100 μm i.d. capillary. C) Microtight union connected to the 30 cm long 100 μm i.d. capillary with 17 cm packed and two 8 mm frits. D) Microtight union connected to 5.5″ capillary with length to the detector of 8 cm.

FIGS. 18A-18B show exemplary set-ups for thermal polymerization using VARIAC VAC transformers and WELLER soldering irons. FIG. 18A shows two VARIAC VAC transformers connected to two soldering irons with 3 mm tips for temperature control. FIG. 18B shows a custom-built hollow aluminum alignment jig used to stabilize the 3 mm tips of the soldering irons during polymerization.

FIGS. 19A-19B show SEM Images of UV and Thermal Polymerized Frits. FIG. 19A shows a frit synthesized with soldering iron thermal polymerization at 92° C. for 75 s.

FIG. 19B shows a frit synthesized with UV polymerization for 1 hr.

FIGS. 20A and 20B shows exemplary set-ups and SEM images for thermal polymerization of the monomer solution to form polymer frits.

### DESCRIPTION OF PREFERRED EMBODIMENTS

Following is a list of elements corresponding to a particular element referred to herein:
5 ligand
10 assay platform
15 microparticle
17 microparticle surface
20 lipid vesicle
22 lipid bilayer
25 receptor
100 apparatus
105 capillary
107 capillary wall
109 capillary end
110 heating device
115 heating tip
120 temperature controlling device
130 jig
132 jig end

As used herein, the term “sorbyl-containing monomer-” refers to a compound containing a 2,4-hexadienoyl group. Non-limiting examples of sorbyl-containing monomers that can be used as polymerizable lipid monomers include 1,2-bis[10-(2′,4′-hexadieoyloxy) decanoyl]-sn-glycero-2-phosphocholine (bis-SorbPC), mono-sorbyl phosphatidylcholine (mono-SorbPC), dienoyl sorbyl phosphatidylcholine (den-SorbPC), and other related compounds such as bi-Sorb and mono-Sorb lipids of different carbon total tail lengths that range from 15,15 to 19,19 and mixtures such as 17,19.

As used herein, the term “a dienoyl-containing monomer” refers to a compound containing a functional group derived from dienoic acid by loss of a hydroxyl group. Non-limiting examples of dienoyl-containing monomers include 1,2-bis(octadeca-2,4-dienoyl)-sn-glycero-3-phosphocholine (bis-DenPC), mono-dienoyl phosphatidylcholine (mono-DenPC), and other related compounds such as bi-Den and mono-Den lipids of different carbon total tail lengths that range from 15,15 to 19,19 and mixtures such as 17,19.

As used herein, the term “stabilized” when used in conjunction with the lipid bilayer means prolonging the lifetime of the lipid bilayer compared to a non-polymerized lipid bilayer. Stabilizing the lipid bilayer does not limit the flexibility of the lipid bilayer, and it still imparts flexibility to allow protein receptors to be embedded in lipid bilayer after polymerization. For example, in one embodiment, the stabilized lipid bilayer is stable for at least 4 h. In other embodiments, the stabilized lipid bilayer is stable for 2 days, 4 days, 7 days, or up to 30 days. As a non-limiting example, stabilized vesicles prepared from sorbyl- and dienoyl-containing lipid monomers are stable for 7-30 days. As another non-limiting example, polymer scaffold vesicles (e.g. vesicles with non-lipid polymerizable monomers) are stable for 2-7 days, but often longer.

### Stabilized Vesicle-Functionalized Microparticles

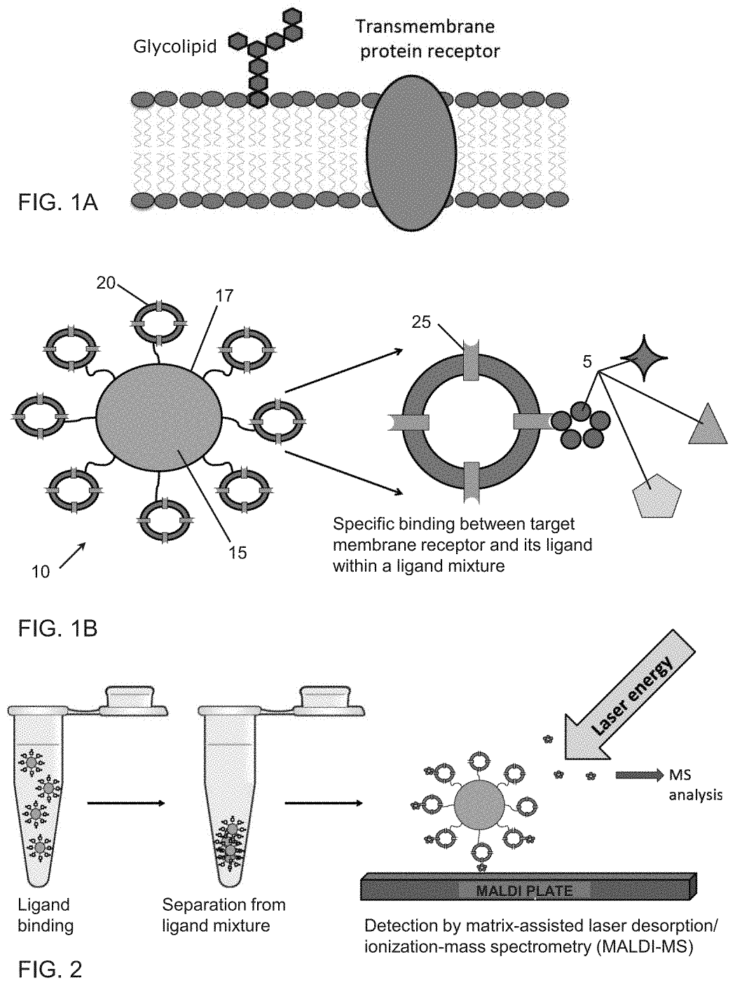

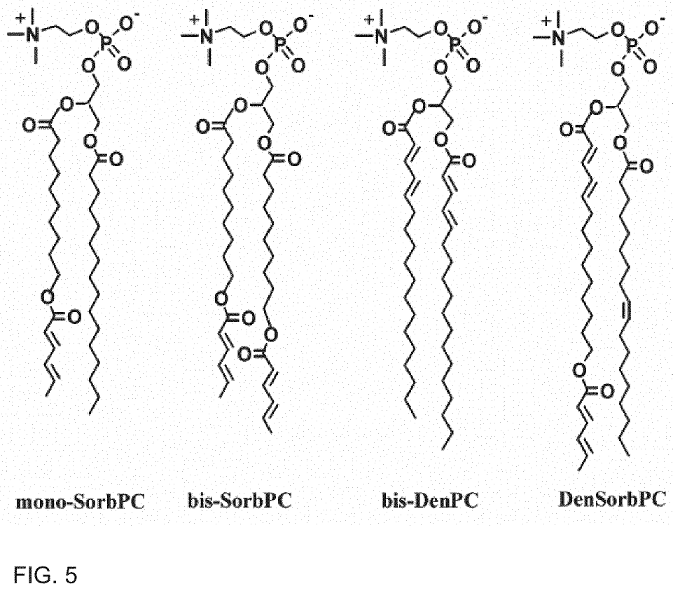

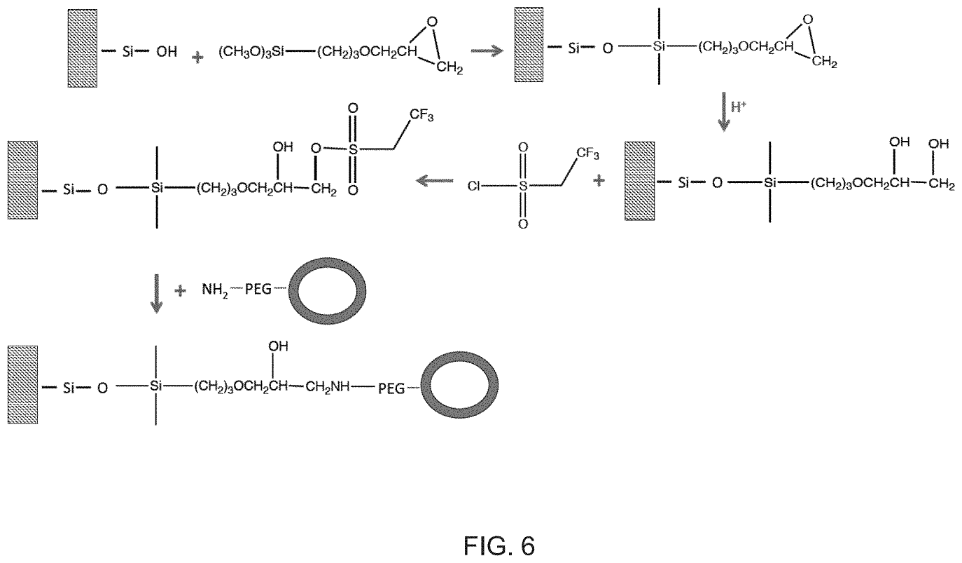

Referring now to FIG. 1-10B, the present invention features a functionalized microparticle for selecting a ligand. In some embodiments, the microparticle comprises a microparticle core, a plurality of lipid vesicles, and one or more target receptors embedded in the lipid bilayers of the vesicles. In further embodiments, each vesicle comprises a lipid bilayer having a polymerized crosslinked structure, and one or more target receptors are specific to the ligand. In some embodiments, the functionalized microparticle is used to select one or more ligands. In further embodiments, the ligands may be known or unknown.

In other embodiments, the microparticle is a silica particle. In further embodiments, the microparticle has a diameter between about 1 to 10 μm, and the vesicles have a diameter of about 100-600 nm. In some embodiments, the vesicles are polymerized by thermal polymerization.

In one embodiment, the lipid bilayer comprises polymerizable lipid monomers and functionalized lipid monomers. In some embodiments, the polymerizable lipid monomers are sorbyl- or dienoyl-containing lipid monomers. Non-limiting examples of the sorbyl- or dienoyl-containing lipid monomers include bis-SorbPC, mono-SorbPC, den-SorbPC, other bi-Sorb and mono-Sorb lipids of different carbon total tail lengths, bis-DenPC, mono-DenPC, and bi-Den and mono-Den lipids of different carbon total tail lengths. In some embodiments, the functionalized lipid monomers are amine-functionalized lipid monomers. In further embodiments, an amine functionality of the amine-functionalized lipid monomers is disposed outwardly and away from the vesicles. Non-limiting examples of the amine functionality include amino(polyethylene glycol) (NH₂-PEG).

In other embodiments, the one or more target receptors are membrane protein receptors or lipid-derived receptors. In some embodiments, the lipid bilayer comprises a plurality of non-polymerizable lipid monomers and a plurality of polymerized, non-lipid monomers. In other embodiments, the non-lipid monomers are hydrophobic. Examples of the lipid monomers include, but are not limited to, cell membrane fragments, phosphatidylcholine monomers, naturally occurring lipids, or synthetic lipids.

In some embodiments, the polymerized crosslinked structure stabilizes the lipid bilayer. In one embodiment, the stabilized lipid bilayer is stable for at least 4 h. In other embodiments, the stabilized lipid bilayer is stable for 2 days, 4 days, 7 days, or up to 30 days. As a non-limiting example, stabilized vesicles prepared from sorbyl- and dienoyl-containing lipid monomers are stable for 7-30 days. As another non-limiting example, polymer scaffold vesicles (e.g. vesicles with non-lipid polymerizable monomers) are stable for 2-7 days, but often longer.

In one embodiment, the surface of the microparticle is modified to provide a covalent attachment point for the lipid bilayer of the vesicles. In further embodiments, the surface of the microparticle is sulfonate-modified such that the surface comprises sulfonate molecules, wherein the sulfonate-modification provides a covalent attachment point for the lipid bilayer of the vesicles. In other embodiments, the present invention features an assay platform for selecting a ligand. The assay platform comprises mixing the functionalized microparticle into a solution comprising the ligand, the ligand binds to a receptor of the one or more target receptors to form a ligand-bound assay platform. The ligand-bound assay platform is removed from the solution, and detected via an analytical instrument, and when the ligand is detected, the ligand is selected by the receptor that is specific to the ligand. In other embodiments, the solution contains one or more ligands. In some embodiments, the one or more ligands may be known or unknown. In further embodiments, the assay platform enables identification of all ligands from the mixture that bind selectively to the receptor. In one embodiment, structural analysis is performed to identify the structure of the one or more ligands.

Another embodiment of the present invention features a method for quantifying a known ligand. The method may comprise providing any embodiment of the assay platform described herein, mixing the assay platform into a solution comprising the ligand such that the ligand binds to the target receptors to form a ligand-bound assay platform, removing the ligand-bound assay platform from the solution, and utilizing an analytical instrument to quantify the specific ligand bound to receptor of the assay platform.

In some embodiments, the present invention features a method for selecting a ligand using a plurality of functional microparticles. The method comprises mixing the plurality of functionalized microparticles with a solution comprising the ligand, where the ligand binds to a receptor of the one or more target receptors to form a ligand-bound receptor. In further embodiments, the method comprises removing the ligand-bound receptor from the solution, and detecting the ligand using an analytical instrument.

Referring now to FIG. 1-10B, the present invention features an assay platform (10) for selecting a ligand (5). In some embodiments, the assay platform (10) may comprise one or more microparticles (15), a plurality of lipid vesicles (20) each comprising a lipid bilayer (22), wherein the vesicles (20) are bonded to a surface (17) of each microparticle, and one or more target receptors (25) specific to the ligands (5). For example, the vesicles (20) may be covalently or non-covalently bonded to the surface (27) of microparticles. Preferably, the receptors (25) are embedded in the lipid bilayer (22) of the vesicle. The assay platform (10) can be mixed into a solution comprising the ligand (5) such that the ligand (5) binds to the target receptor (25) to form a ligand-bound assay platform. The ligand-bound assay platform is removed from the solution, and can be detected via an analytical instrument. When the ligand (5) is detected, the ligand (5) can be selected by the receptor (25) that is specific to the ligand (5). For example, the unknown ligand can be identified if it binds to the receptors since the identity of the receptors is known and the receptors are specific to the ligand.

In some embodiments, the microparticles (15) are silica particles. A diameter of each microparticle can range from between about 1 to 10 μm. In other embodiments, the vesicles (20) may be spherical in shape and have a diameter of about 100-600 nm. In some embodiments, the receptors (25) can be membrane protein receptors or lipid-derived receptors.

In one embodiment, the lipid bilayer (22) may comprise polymerizable lipid monomers and functionalized lipid monomers. The lipid bilayer (22) of the vesicles (20) can be polymerized by thermal polymerization. The polymerizable lipid monomers may be sorbyl- or dienoyl-containing lipid monomers. As non-limiting examples, the sorbyl- and dienoyl-containing lipid monomers are bis-SorbPC, mono-SorbPC, den-SorbPC, other bi-Sorb and mono-Sorb lipids of different carbon total tail lengths, bis-DenPC, mono-DenPC, and bi-Den and mono-Den lipids of different carbon total tail lengths. In other embodiments, the functionalized lipid monomers are amine-functionalized lipid monomers. For example, the amine-functionalized lipid monomers may comprise an amino(polyethylene glycol) (NH₂-PEG) component. Preferably, the amine functionality of the amine-functionalized lipid monomers is disposed outwardly and away from the vesicle (20), thereby making the microparticles (15) amine-reactive. In some embodiments, the mole ratio of the polymerizable lipid monomers, functionalized lipid monomers, and receptors may be about 95:5:1.

In other embodiments, the lipid bilayer (22) may comprise a plurality of non-polymerizable lipid monomers and a plurality of polymerized, hydrophobic non-lipid monomers. For example, the lipid monomers may be cell membrane fragments, 1,2-diphytanoyl-sn-glycero-3-phosphocholine monomers, naturally occurring lipids, or synthetic lipids. In some embodiments, the plurality of polymerized, hydrophobic non-lipid monomers may comprise a methacrylate and a cross-linking agent. The methacrylate may be an aliphatic methacrylate, such as an alkyl-substituted aliphatic methacrylate having an alkyl substitution of C4-C10, or an aromatic methacrylate, such as a benzyl methacrylate or a naphthyl methacrylate. The cross-linking agent can be a dimethacrylate, such as ethylene glycol dimethacrylate.

In preferred embodiments, the surface (17) of each microparticle may be surface-modified, such as sulfonate-modification. Without wishing to limit the present invention to a particular theory or mechanism, the surface-modification can provide a covalent attachment point for the lipid bilayer (22) of each vesicle. For example, the surface (17) may be sulfonate modified such that the surface (17) comprises sulfonate molecules. Examples of sulfonates that may be used in accordance with the present invention include, but are not limited to, 2,2,2-trifluoroethanesulfonyl chloride, alkyl p-toluenesulfonates (tosylates) and related compounds. Sulfonate-modification is but one example of surface modification. It is to be understood that the surface of the microparticle may be modified with any suitable molecule that can provide a covalent attachment point for the lipid bilayer of each vesicle.

Another embodiment of the present invention features a method for selecting a ligand (5). The method may comprise providing any embodiment of the assay platform (10) described herein, mixing the assay platform (10) into a solution comprising the ligand (5) such that the ligand (5) binds to the target receptors (25) to form a ligand-bound assay platform, removing the ligand-bound assay platform from the solution, and utilizing an analytical instrument to detect the ligand bound to receptor of the assay platform. When the ligand (5) is detected, the ligand (5) is selected by the receptor (25) that is specific to the ligand (5).

A further embodiment of the present invention features a method of preparing an assay platform (10) for selecting a ligand (5). The method may comprise providing a plurality of microparticles (15), depositing modifying molecules on a surface (17) of each microparticle to form a surface-modified microparticle, mixing a plurality of lipid monomers with one or more target receptors specific to the ligand to yield a monomer-receptor mixture, forming the monomer-receptor mixture into a plurality of lipid vesicles (20) such that each lipid vesicle has a lipid bilayer (22), polymerizing the lipid vesicles (20) such that the receptors (25) are embedded in the lipid bilayer (22) of each vesicle, and depositing the vesicles (20) on the surface of each surface-modified microparticle to form the assay platform (10).

In one embodiment, the microparticles (15) are silica particles. A diameter of each microparticle can range from between about 1 to 10 μm. In another embodiment, the vesicles (20) are polymerized by thermal polymerization. The vesicles can have a diameter of about 100-600 nm. In yet another embodiment, the monomer-receptor mixture is formed into a plurality of lipid vesicles (20). A non-limiting example of forming said lipid vesicles utilizes surfactant dialysis. The receptor is solubilized into a solution of surfactants that are subsequently removed by dialysis in the presence of excess lipid vesicles to localize the receptor into the lipid vesicle membrane.

In some embodiments, when the receptors (25) are membrane protein receptors, the method may further comprise reconstituting the membrane protein receptor with a surfactant prior to polymerizing the lipid vesicles. In alternative embodiments, the receptors (25) are lipid-derived receptors.

In some embodiments, the lipid monomers comprise polymerizable lipid monomers and functionalized lipid monomers. The polymerizable lipid monomers may be sorbyl- or dienoyl-containing lipid monomers. As non-limiting examples, the sorbyl- and dienoyl-containing lipid monomers are bis-SorbPC, mono-SorbPC, den-SorbPC, other bi-Sorb and mono-Sorb lipids of different carbon total tail lengths, bis-DenPC, mono-DenPC, and bi-Den and mono-Den lipids of different carbon total tail lengths. In other embodiments, the functionalized lipid monomers are amine-functionalized lipid monomers. For example, the amine-functionalized lipid monomers may comprise an amino(polyethylene glycol) (NH₂-PEG) component. Preferably, the amine functionality of the amine-functionalized lipid monomers is disposed outwardly and away from the vesicle (20), thereby making the microparticles amine-reactive. As a non-limiting example, a mole ratio of the polymerizable lipid monomers, functionalized lipid monomers, and receptors may be about 95:5:1.

In some embodiments, the method may further comprise mixing a plurality of polymerizable, hydrophobic non-lipid monomers with the monomer-receptor mixture prior to polymerizing the mixture. The lipid monomers may be cell membrane fragments, 1,2-diphytanoyl-sn-glycero-3-phosphocholine monomers, naturally occurring lipids, or synthetic lipids. The plurality of polymerizable, hydrophobic non-lipid monomers may comprise a methacrylate and a cross-linking agent. In one embodiment, the methacrylate is an aliphatic methacrylate, such as an alkyl-substituted aliphatic methacrylate having an alkyl substitution of C4-C10, or an aromatic methacrylate, such as benzyl methacrylate or a naphthyl methacrylate. In another embodiment, the cross-linking agent is a dimethacrylate, such as ethylene glycol dimethacrylate.

In other embodiments, the modifying molecules may be sulfonate molecules, which can provide covalent attachment points for the lipid bilayer of the vesicles to attach to the surface-modified microparticle. Examples of sulfonates that may be used in accordance with the present invention include, but are not limited to, 2,2,2-trifluoroethanesulfonyl chloride, tosylates, and related compounds. It is to be understood that the present invention is not limited to sulfonate modification, and that the surface of the microparticle may be modified with any suitable molecule that can provide covalent attachment points.

An exemplary embodiment of the present invention features a microparticle architecture that utilizes a silica core particle (15) that is functionalized with receptors within stabilized vesicles (or liposomes). This particle architecture was then used to perform pulldown assays in complex solutions with subsequent analysis by flow cytometry. Recovery of serotonin via binding to 5-HT1A receptors within CHO-K1 cell membranes was evaluated. CHO-K1 cell membrane fractions were isolated through homogenization and centrifugation, and were extruded to form vesicles (20), which could be subsequently stabilized using polymer scaffold stabilization approaches. The vesicles (20) were then immobilized to the particle surface (17) to yield silica core-cell membrane vesicle shell particles. Particles were characterized using flow cytometry to verify attachment of cell membrane vesicles with and without 5-HT1A receptors to modified particles. Serotonin was incubated with the silica core-cell membrane vesicle shell particles containing the serotonin receptor, and centrifugation was used to pull down the particles. Flow cytometry confirmed the pull down of the serotonin ligand.

### Experimental Procedures

The following are exemplary embodiments of preparing silica core-vesicle shell particles and detecting peptide/protein ligands on specific targets embedded in the particles. It is understood that the present invention is not limited to the embodiments described herein. For instance, the following example uses bis-SorbPC as the polymerizable lipid monomer. However, bis-SorbPC may be substituted for mono-SorbPC, den-SorbPC, other bi-Sorb or mono-Sorb lipids of different carbon total tail lengths, bis-DenPC, mono-DenPC, or bi-Den or mono-Den lipids of different carbon total tail lengths.

Sulfonate Modification of Silica Particles:

Procedures are modified from Larson (Methods Enzymol. 1984, 104, 212-223) and Nilsson (Methods Enzymol. 1984, 104, 56-69).

Diol-Modification of Silica Particles:

About 40 mg of 5 μm diameter silica particles was suspended in 20 mL of 5% HCl and stirred for 1 hour. The particles were then washed with nanopure water three times and with acetone three times sequentially by centrifugation. After washing, silica particles were re-suspended in a small amount of acetone and transferred into a 50 mL round bottom flask. The acetone was evaporated by a stream of N₂, and the flask was connected to a vacuum and heated to 150° C., lasting for 4 hours. After 4 hours, heating was stopped. When the temperature dropped to between about 50-100° C., the vacuum was disconnected. A stir bar was placed into the flask. Then a mixture of 30 mL dry toluene, 0.6 mL 3-glycidyloxypropyltrimethoxysilane and 15.3 μL triethylamine was added into the flask. The flask was connected to a condenser, which was sealed with a septum. The system was flushed with N₂ three times and then an N₂ balloon was attached to the top of the condenser. The mixture was heated to reflux, which lasted overnight. After overnight reflux, the particles were washed sequentially with toluene and acetone, and then dried with a stream of N₂. Dried particles were re-suspended in 20-30 mL of 10 mM H₂SO₄ (by sonication) and then heated to 90° C. for 1 hour with stirring. After that, the particles were washed sequentially with water and acetone.

Sulfonate Modification of Diol-Silica Particles:

Diol-silica particles were washed with dry acetone three times. Then the particles were transferred into a cleaned, dried, 25 mL round bottom flask. The remaining dry acetone was evaporated with an N₂ stream and a stir bar was put inside the flask. The flask was sealed with a septum and flushed with N₂ for several minutes. About 3 mL of dry acetone was added into the flask and stirring was started. Then 26 μL dry pyridine and 18 μL 2,2,2-trifluoroethanesulfonyl chloride were added sequentially. The mixture was stirred for 15-30 min at room temperature. After the reaction, sulfonate-silica particles obtained were washed with acetone, 1:1 (v:v) of acetone:5 mM HCl, 1 mM HCl, nanopure water and acetone sequentially. Lastly, the particles were dried with N₂ stream and stored dry.

Aside from sulfonate modification, other surface modification chemistries can be used, such any appropriate modifications using covalent and non-covalent linkages of vesicles to the surface.

Formation and Thermal Polymerization of Bis-sorbPC/NH/GM1 Vesicles:

Bis-sorb PC was purified by HPLC as described in Gallagher (J. Chromatogr. A. 2015, 1385, 28-34). Procedures for polymerization of bis-sorbPC vesicles were modified from Sisson (Macromolecules. 1996, 29, 8321-8329). About 0.95 mg bis-sorbPC was mixed with 1,2-distearoyl-sn-glycero-3-phosphoethanolamine-N-[amino(polyethylene glycol)-2000] (DSPE-PEG (2000)-NH₂) and GM1 to make a mole ratio of about 95:5:1 of bis-sorbPC:GM1:DSPE-PEG (2000)-NH₂. Azobisisobutyronitrile (AIBN) was dissolved in benzene to make fresh stock solution of 1 mg/ml. An appropriate amount of ABN stock was added to the lipid mixture to make a mole ratio of 2.5:1 bis-sorbPC:AIBN. Organic solvents in the mixture were evaporated with an Ar stream and the mixture was further dried in a lyophilizer for at least 4 hours. After drying, the lipid mixture was re-hydrated in 200 μL of degassed 20 mM phosphate, pH 7.4. The sample was warmed up in a 42° C. water bath and vortexed to re-suspend all the dried lipids. Then the sample went through 10 cycles of freeze (−77° C.), thaw (42° C.), vortex, and was extruded through 2 stacked 0.2 μm polycarbonate membrane filters by a mini extruder. The extrusion was carried out above the Tₘ (29° C.) of bis-sorbPC. After extrusion, the vesicle solution was bubbled with a slight stream of Ar for 5 min and sealed with an Ar atmosphere. Then the vesicle solution was heated at 65° C. overnight for thermal polymerization. The handling of bis-sorbPC was done under yellow light before polymerization.

Formation of Silica Core-Vesicle Shell Particles:

About 200 μL of 20 mM phosphate, pH 8.0 was added to 4 mg of sulfonate-silica particles. The mixture was sonicated to re-suspend the particles. The 200 μL of polymerized vesicle solution was then added into the particle suspension. The total 400 μL of sample was placed into 0.2 mL dome cap PCR tubes to completely fill the tube and cap space and eliminate air. The sample was incubated by slowly inverting up and down continuously on a mixing wheel for 3 hours. After incubation, samples were centrifuged at 7 G for 5 min and the supernatant was discarded. After tethering vesicles to the sulfonate-silica particles, washing of the particles by centrifugation needs to be done at low G forces to minimize loss of vesicle coating on the particle surface. About 400 μL of 20 mM phosphate, 5 mM Tris, pH 8.0 was added to the particles, and the inverting incubation was continued for 1 hour to scavenge the sulfonate-silica surface that was not covered by vesicles. After surface scavenge, the particles were washed three times by 20 mM phosphate, pH 7.4 (7 G×5 min each time).

Detection and Identification of CTB with MALDI-MS:

After the silica core-vesicle shell particles were washed by 20 mM phosphate, pH 7.4, the supernatant was discarded. Then about 114 μL of 0.5 mg/mL CTB stock solution was added to the particles, and an appropriate amount of 20 mM phosphate, pH 7.4 was added to the mixture to completely fill the 0.2 mL dome cap PCR tubes. The inverting incubation was carried out for 1 hour. Then the particles were washed three times by 20 mM phosphate, pH 7.4 (7 G×5 min each time). Lastly, the particles were re-suspended in 200 μL of 20 mM phosphate, pH 7.4. About 1 μL from the particle suspension was mixed with 1 μL of saturated CCA in water. The total 2 μL of mixed sample was spotted on MALDI plate and dried at room temperature. MALDI-MS analysis was directly carried out on the dried particles. A Bruker UltraFlex III TOF-TOF mass spectrometer was operated in linear, positive ion mode.

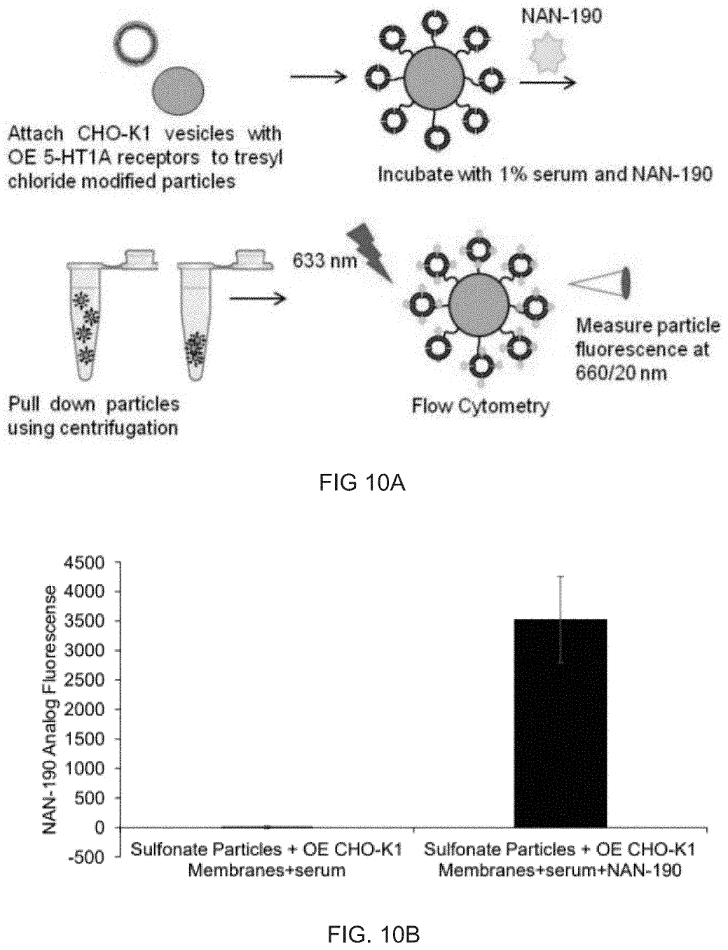

Referring to FIGS. 10A and 10B, the following is a non-limiting example of preparing silica core-vesicle shell particles functionalized with transmembrane receptors and detecting peptide/protein ligands on specific targets embedded in the particles.

As shown in FIG. 10A, the buffers used were 50 mM Tris-HCl, pH 7.4, 10 mM MgSO₄, 0.5 mM EDTA, 0.1% ascorbic acid (Buffer A), and 50 mM Tris-HCl, pH 7.4 (Buffer B). In one embodiment, 5 μm silica particles were modified to obtain sulfonate surface modification. In another embodiment, the vesicles were CHO-K1 cell membranes with overexpressed 5-HT1A receptors extruded through 200 nm filters. About 0.5 mg CHO-K1 cell membranes with overexpressed 5-HT1A receptors. OE-CHO-K1 were attached to 0.5 mg of sulfonate modified particles through incubation for 3 hrs while spinning on a bloodwheel, thereby forming the silica core-vesicle shell particles. Then, 1% serum (final concentration) was added to both samples. Fluorescent 5-HT1A receptor antagonist, NAN-190 was added to sample 2 and incubated for 1 hour. Samples were washed 3 times with Buffer B. Two samples were prepared: Sample 1—OE-CHO-K1 Membrane+1% FBS serum; and Sample 2—OE CHO-K1 Membrane+1% FBS serum+NAN-190. 10,000 particles from each sample were analyzed using flow cytometry λₑₓ: 633 nm λₑₘ:660/20 nm.

Referring to FIG. 10B, the silica core-shell particles with CHO-K1 cell membranes vesicles overexpressing the 5-HT1A receptor successfully bound NAN-190 in the presence of serum. Without wishing to limit the invention to a particular theory or mechanism, the G-protein coupled receptor is proven to have remained functional in this new platform due to the specific binding of NAN-190.

### Results and Discussion

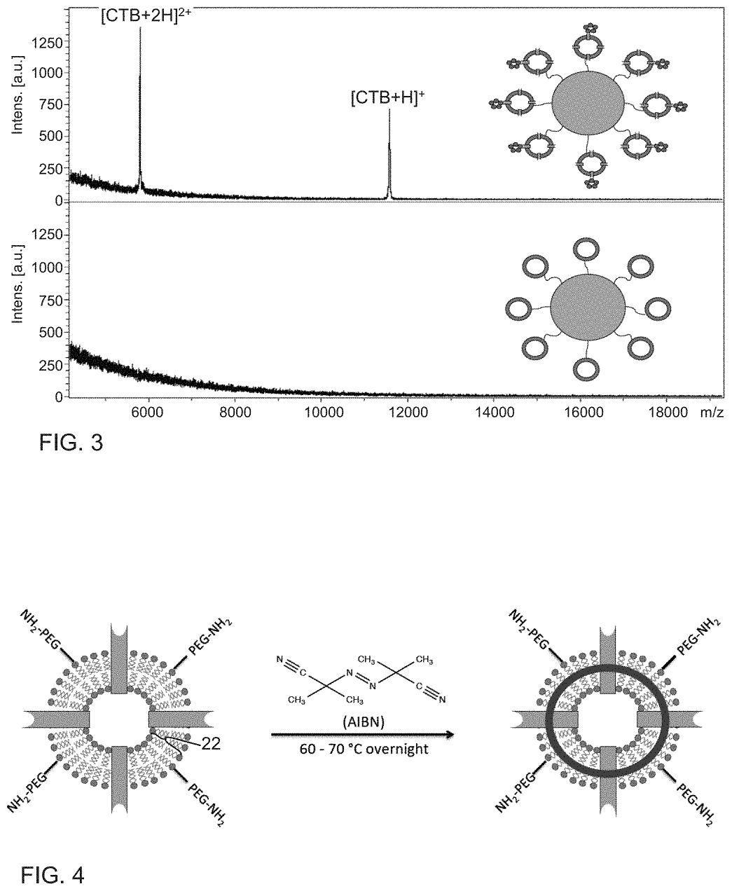

Referring to FIG. 3, cholera toxin binding unit (CTB) was successfully detected using ganglioside GM1 (CTB's membrane receptor) functionalized core-shell particles. The top mass spectrum shows CTB molecular ions (both singly charged and doubly charged) were successfully detected with MALDI-TOF-MS analysis on the silica core-polybis-sorbPC/NH₂/GM1 vesicle shell particles, after incubating with CTB and after washing, because of the specific binding between GM1 and CTB. The bottom mass spectrum shows that no CTB was detected on the silica core-polybis-sorbPC/NH₂ vesicle shell particles, after incubating with CTB and after washing, because of the lacking of GM1.

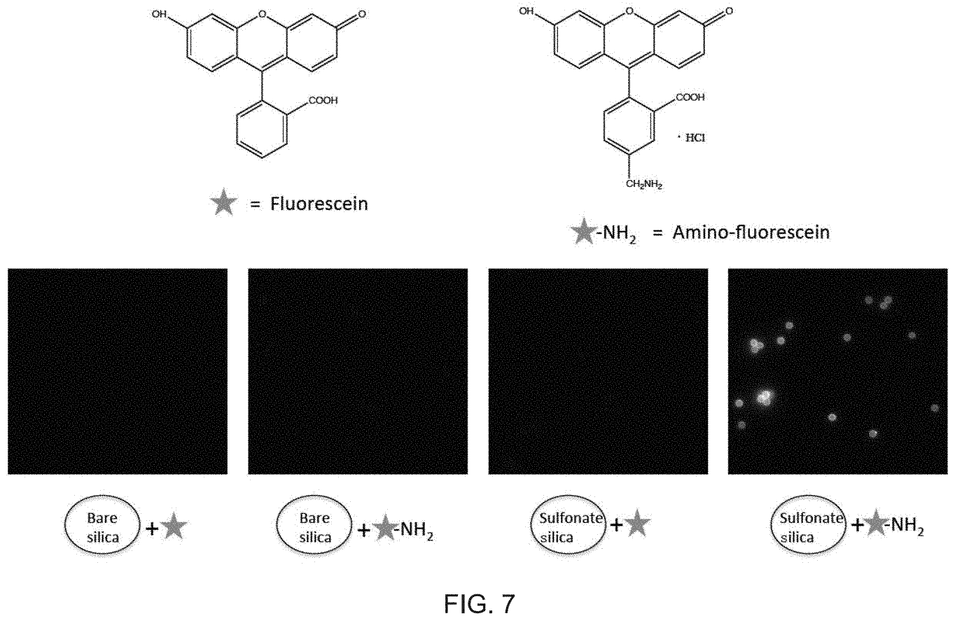

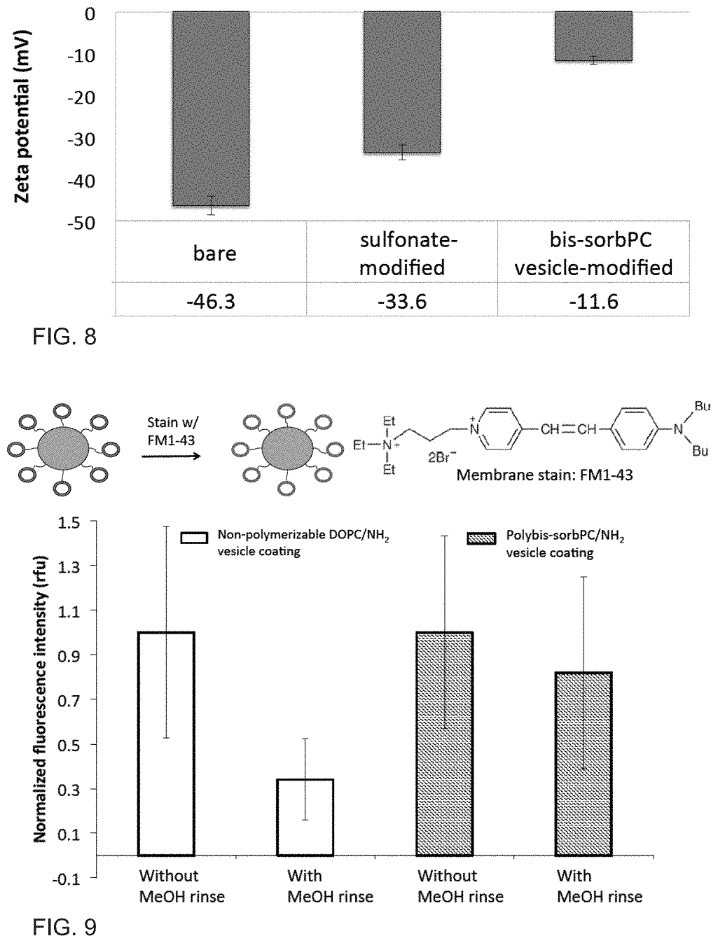

Referring to FIG. 7, the fluorescence image on the far right shows that the sulfonate-modified silica microspheres may be amine-reactive. As shown FIG. 8, the phospholipid vesicle coating may change the surface charge of the silica microspheres. A sulfonate-modified microsphere has a smaller negative zeta potential than that of a bare microsphere. Further still, a bis-sorbPC vesicle-modified microsphere has an even lower negative zeta potential than that of the sulfonate-modified microsphere or the bare microsphere. As shown in FIG. 9, after a MeOH rinse, polybis-sorbPC/NH₂ vesicle coated microspheres have a normalized fluorescence intensity that is about 2.5 times greater than that of non-polymerizable DOPC/NH₂ vesicle coated microspheres. This demonstrates that the polymerized vesicle coatings are more stable than non-polymerizable vesicle coatings on sulfonate-modified silica microspheres.

When synthesizing the silica core-polymerized phospholipid vesicle shell particles of the present invention, polymerization of the phospholipids was shown to improve vesicle coating stability. The present invention allows for peptides or protein ligands to be detected on specific targets, such as receptors, of the functionalized core-polymerized phospholipid vesicle shell particles. Examples include, but are not limited to, the detection of CTB on GM1-functionalized core-shell particles by MALDI-MS and the detection of serotonin via binding to 5-HT1A receptors. The silica core-shell particles with immobilized cell membranes vesicles containing membrane receptors of the present invention may be used as a simple and fast approach for high throughput ligand screening.

### Rapid Formation of Polymer Frits in Capillaries

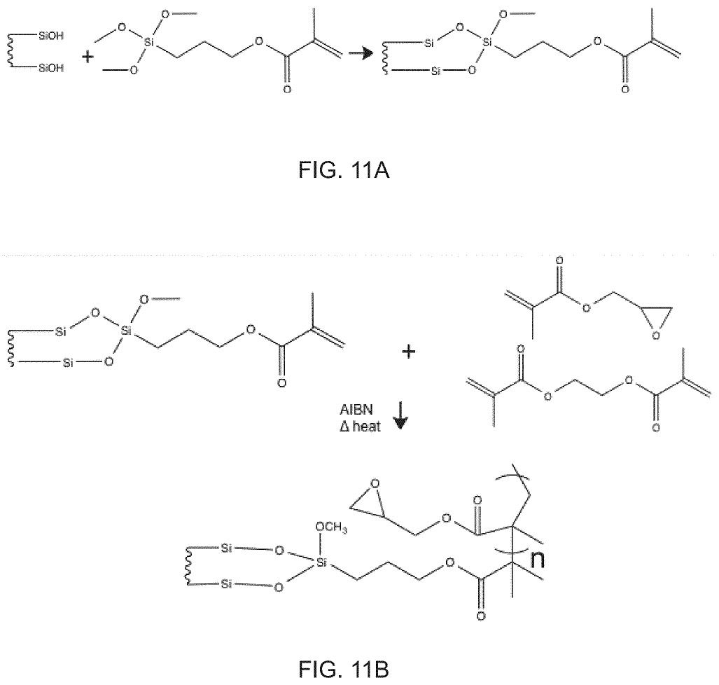

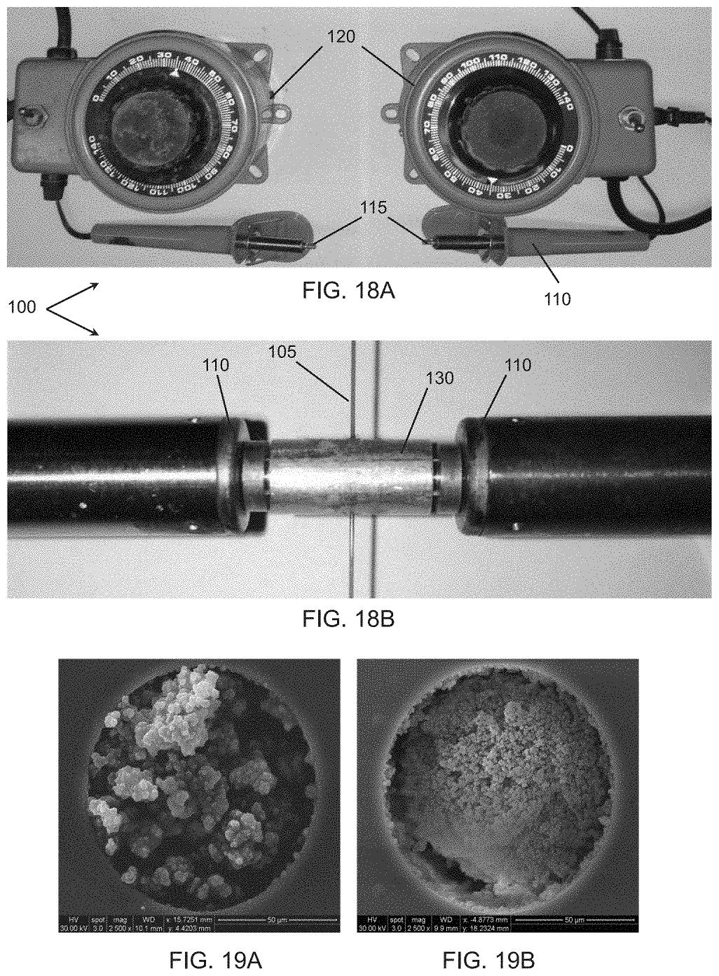

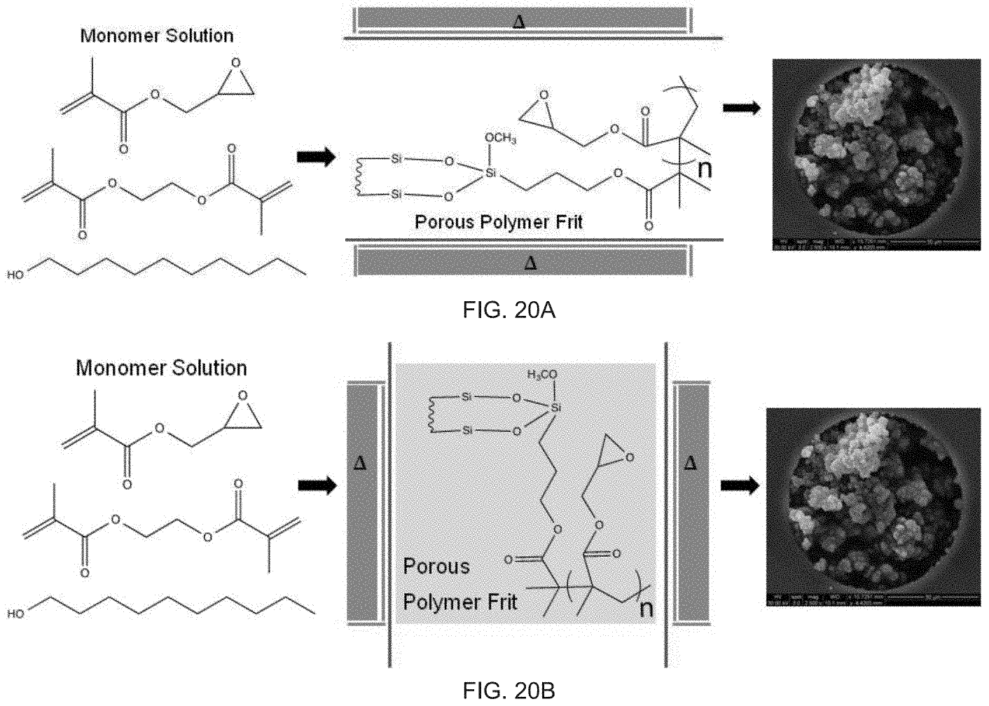

Referring now to FIG. 11-20B, the present invention features a method of forming polymer frits inside a capillary (105). In one embodiment, the method may comprise providing one or more heating devices (110) each having a heating tip (115), wherein the heating devices (110) are operatively connected to one or more temperature controlling devices (120), connecting each heating tip (115) together via a jig (130), rinsing the capillary (105) with a monomer solution such that at least a portion of the capillary is filled with the monomer solution, heating the heating tips (115) via the heating devices (110) to a polymerization temperature set by the temperature controlling devices (120), inserting the capillary (105) through the jig (130) such that the portion of the capillary containing the monomer solution is disposed between the heating tips (115), and heating the portion of the capillary via the heating tips (115) for a polymerization time to thermally polymerize the monomer solution, thereby forming the polymer frits, thereby forming the polymer frits.

Without wishing to limit the present invention to a particular theory or mechanism, the method can be effective for placement of the polymer frits at a spatially-defined location in the capillary (105), i.e. the heating tips can polymerize the monomer solution into polymer frits at precise locations along the capillary (105). Moreover, thermal polymerization can retain a capillary sheath of the capillary, thereby improving capillary stability, as opposed to other methods of polymerization where the capillary sheath is removed.

As used herein, the term “frit” refers to a porous material with pore sizes sufficiently small enough to retain particles, e.g. a fused or partially fused porous material.

As used herein, the term “jig” is defined as a device that is used to control a location and/or motion of other tools, or parts thereof.

In some embodiments, the heating devices (110) may comprise a soldering iron and the heating tip (115) is a soldering tip. In other embodiments, any appropriate heating device may be used to heat the capillary. In further embodiments, the temperature controlling devices (120) may comprise a variable alternating current transformer. However, it is understood that the temperature controlling devices (120) may also be any appropriate device that controls temperature. For example, the soldering iron may be an adjustable temperature soldering iron. In still other embodiments, the jig (130) is a metallic tubular jig. For example, the jig may be constructed from a metal such as aluminium, steel, iron, or any other suitable metal or metal alloy. Each heating tip (115) is positioned at a jig end (132) of the jig such that the heating tip is disposed in the jig. For instance, the heating tip is inserted into the jig end. Preferably, the capillary (105) is perpendicularly disposed through the jig (130).

In some embodiments, the polymerization temperature is about 92-140° C. For example, the polymerization temperature may be about 90-100° C., 100-120° C., 120-140° C., or greater than 140° C. In other embodiments, the polymerization time is about 10-110 seconds. For instance the polymerization time may be about 10-30 seconds, 30-60 seconds, 60-90 seconds, or 90-110 seconds. Preferably, the polymerization time is less than 2 minutes.

Embodiments of the present invention may utilize a fused silica capillary. The capillary (105) may be modified prior to rinsing with the monomer solution. In exemplary embodiments, the step of modifying the capillary comprises rinsing the capillary (105) with a modifying solution comprising methacrylate under UV-free yellow light, wherein at least a portion of the capillary (105) is filled with the modifying solution, capping each capillary end (109) to seal the modifying solution within the capillary (105), heating the capillary (105) at a temperature of 60° C. for about 15 to 20 hours, and removing the modifying solution from the capillary (105). Preferably, the methacrylate can modify a capillary wall (107) to increase stability when the polymer frits are bonded to the capillary wall. The polymer frits may be secondary frits for retaining a packed-bed inside the capillary (105). In some embodiments, the capillary (105) may be dried after the modifying solution is removed. In other embodiments, each capillary end (109) may be capped.

In some embodiments, the methacrylate may be 3-trimethyoxysilylpropyl methacrylate (TMSPM). However, any suitable methacrylate may be used to modify the capillary wall. In other embodiments, the monomer solution may comprise glycidyl methacrylate (GMA), ethylene glycol dimethacrylate (EGDMA), and 2,2-azobisisobutyronitrile. An example of the volumetric ratio of GMA to EGDMA may be a 1:1 volumetric ratio. The monomer solution may also further comprise decanol.

Another embodiment of the present invention apparatus (100) for forming polymer frits inside a capillary (105). The apparatus may comprise one or more heating devices (110) each having a heating tip (115), wherein the heating devices (110) are operatively connected to one or more temperature controlling devices (120), and a tubular jig (130). In some embodiments, each heating tip (115) can be each positioned at a jig end (132) such that the heating tip (115) is disposed in the jig (130). The heating tips are heated via the heating devices to a polymerization temperature set by the temperature controlling. The capillary (105) may be perpendicularly disposed through the jig (130) such that the portion of the capillary (105) containing the monomer solution is positioned between the heating tips (115), which then thermally polymerizes the monomer solution to form the polymer frits. Preferably, the apparatus may be effective for placement of the polymer frits at a spatially-defined location in the capillary.

In some embodiments, the heating devices (100) may comprise a soldering iron and the heating tip (115) is a soldering tip. In other embodiments, any appropriate heating device may be used to heat the capillary. In further embodiments, the temperature controlling devices (120) may comprise a variable alternating current transformer. However, the temperature controlling devices may also be any appropriate device that controls temperature. For example, the soldering iron may be an adjustable temperature soldering iron. In still other embodiments, the jig (130) may be constructed from a metal, such as aluminum, steel, iron, or any other suitable metal or metal alloy.

In some embodiments, the polymerization temperature may be about 92-140° C. For example, the polymerization temperature may be about 90-100° C., 100-120° C., 120-140° C., or greater than 140° C. In other embodiments, the polymerization time is about 10-110 seconds. For instance the polymerization time may be about 10-30 seconds, 30-60 seconds, 60-90 seconds, or 90-110 seconds. Preferably, the polymerization time is less than 2 minutes.

Examples of capillaries (105) may be silica capillaries and fused silica capillaries. Preferably, a capillary wall (107) of the capillary is modified with a methacrylate to effect bonding of the polymer frits to the capillary wall for increased stability. Examples of the methacrylate include 3-trimethyoxysilylpropyl methacrylate (TMSPM) or any other suitable methacrylate.

In some embodiments, the monomer solution may comprise glycidyl methacrylate (GMA), ethylene glycol dimethacrylate (EGDMA), and 2,2-azobisisobutyronitrile. An example of the volumetric ratio of GMA to EGDMA may be a 1:1 volumetric ratio. In other embodiments, the monomer solution may further comprise decanol.

### EXPERIMENTAL

Materials:

Fused silica capillary (100 μm i.d., 360 μm o.d.) was purchased from PolyMicroTechnologies. NaOH, HCl, acetone, decanol, HPLC grade methanol (99.9%), ethanol, and n-octylamine (98%) were purchased from Fisher Scientific. 3-trimethyoxysilylpropyl methacrylate (TMSPM), ethylene glycol dimethacrylate (98%) (EGDMA), 2-2-dimethoxy-2-phenylactetophenone (99%) (DAP), and 2,2-azobisisobutyronitrile (97%) (AIBN) were purchased from Sigma Aldrich. AIBN was recrystallized in methanol prior to use to remove impurities. Glycidyl methacrylate (98%) (GMA) was purchased from Alfa Aesar. Trifluoroacetic acid (TFA) and acetonitrile (ACN) were purchased from EMD Millipore. HxSil 5 μm diameter C₁₈-modified silica particles with 100 Å pores were purchased from Hamilton. A Cheminert injection valve was purchased from Valco. N-hexylamine (99%) was purchased from VWR International. Unless noted all chemicals are used as received. All H₂O was purified to a resistivity of 18.3 MO-cm using a Barnstead EASYpure UV/UF compact reagent grade water system.

Capillary Modification:

Sections of fused silica capillary (100 μm i.d., 360 μm o.d.) 30 cm long were rinsed at 0.5 mL min⁻¹ with 1M NaOH (5 min), 0.1M HCl (5 min), nanopure H₂O (5 min), and acetone (10 min) using a syringe pump. Capillaries were dried using He for 30 min. A 50% (v/v) mixture of TMSPM and acetone was rinsed through capillaries under UV-free yellow light. Capillary ends were sealed with parafilm to withhold the solution and heated at 60° C. for 20 h. Capillaries were rinsed with methanol at 1 mL min⁻¹ for 10 min using a syringe pump. Capillaries were dried with He for 30 min and left to further air-dry overnight. This methacrylate modification allows for bonding of the frit to the capillary wall for increased stability.

Monomer Solution Preparation:

GMA and EGDMA were passed through an aluminum oxide column (0.25″×3.0″) prior to use to remove inhibitors. A monomer solution for thermal polymerization was prepared using 1.3 mg AIBN, 60 μL GMA, 60 μL EGDMA, and 280 μL decanol. For UV polymerization, the thermal initiator AIBN was replaced with 2.5 mg DAP while maintaining the other reagents. The monomer solutions were sonicated for 10 min and degassed with N₂ for 10 min.

Frit Synthesis:

Two soldering irons were connected to variable autotransformers to enable temperature control. In-house fabricated 3 mm soldering iron tips were attached to both soldering irons. The VARIAC VAC transformers were adjusted to achieve the desired polymerization temperature ranging from 92-140° C. and turned on 30 min prior to polymerization to allow the soldering irons to equilibrate to the set temperature. The monomer solution was rinsed through capillaries. Capillaries were placed in a custom-fabricated 7 mm long aluminum alignment jig and capillary ends were sealed with parafilm to contain the monomer solution within the capillary. Frits were synthesized by placing a soldering iron on either side of the capillary within the aluminum jig for the designated polymerization time ranging from 10-110 s. The reaction chemistry for frit polymerization is shown in FIGS. 11A and 11B. Capillaries were washed with methanol at 5 μL min⁻¹ for 10 min to remove any unreacted components post polymerization using a syringe pumping system and dried overnight. Frits were imaged using a camera through the lens of a stereomicroscope and measured using Image J software.

UV initiated frits were polymerized in capillaries with 8 mm windows for 1 h using a Newport 100 W Mercury Arc lamp with H₂O IR absorption filter and UV bandpass filter. All UV frits had an average length of 8.0±0.5 mm. UV initiated frits necessitated burning an 8 mm window within the capillary prior to capillary modification.

Frit Pressure Stability Studies:

The equilibration pressure of both thermal and UV initiated frits was monitored at various flow rates. The deviation pressure was defined as a deviation in linearity of pressure versus flow rate corresponding to the onset of frit compression or decomposition.

Packing Capillaries:

An air driven fluid pump was used to pack a slurry solution by pumping methanol at 500 psi. The slurry solution contained 5 μm diameter C₁₈ modified silica particles at a concentration of 6.4 mg particles/mL methanol. A 17 cm bed was packed. To synthesize the second retaining frit, the particle slurry solution was replaced with the monomer solution and pumped through the capillary at 500 psi for 10 min. The second retaining frit was polymerized by using soldering irons to apply heat at the edge of the packed bed or by positioning the previously burned capillary window in front of the lamp for UV irradiation. Packed capillaries were rinsed with methanol at 0.5 μl min⁻¹ for 30 min and dried overnight.

Capillary Liquid Chromatography:

An EldexMicroPro pump equipped with 2 mL syringes was used with a Cheminert injection valve and 1.4 μL injection loop (FIG. 17). A section of 4.5″ 250 μm i.d. PEEK tubing was connected to the outlet of the pump and connected to a 3.5″ long 100 μm i.d. capillary using a Microtight PEEK connector from IDEX. The capillary was connected to the injection valve and a 4″ long 100 μm i.d. capillary. The 30 cm long 100 μm i.d. capillary with a 17 cm packed bed and two 8 mm frits was connected using a Microtight union from Sigma Aldrich to a 5.5″ capillary with length to the detector of 8 cm. A 3 mm detection window was used. 250 μm i.d., 1/16″ PEEK tubing was purchased from GRACE. Zonal chromatography was performed using 125 nM FITC labeled n-hexylamine and n-octylamine dissolved in ethanol. The 45/55 ACN/H₂O 0.1% TFA (v/v) mobile phase was degassed using He for 30 min prior to use. The elution profile was monitored by UV absorbance detection at 240 nm. Signal from the detector was collected with an A/D converter and software written in Lab View. Sovitsky-Golay filtering was applied to the data using Origin.

Scanning Electron Microscopy:

An FEI Inspec-S SEM instrument was used to image both thermal and UV polymerized frits. Prior to imaging, capillaries were mounted vertically and a 4-5 mm gold coating was sputtered onto ends using the Hummer Sputter System. Samples were coated for 90 s, rotated and coated for an additional 90 s.

Safety Considerations:

When packing capillaries, the entire system should be contained to prevent injury if a fitting failed.

### Results and Discussion

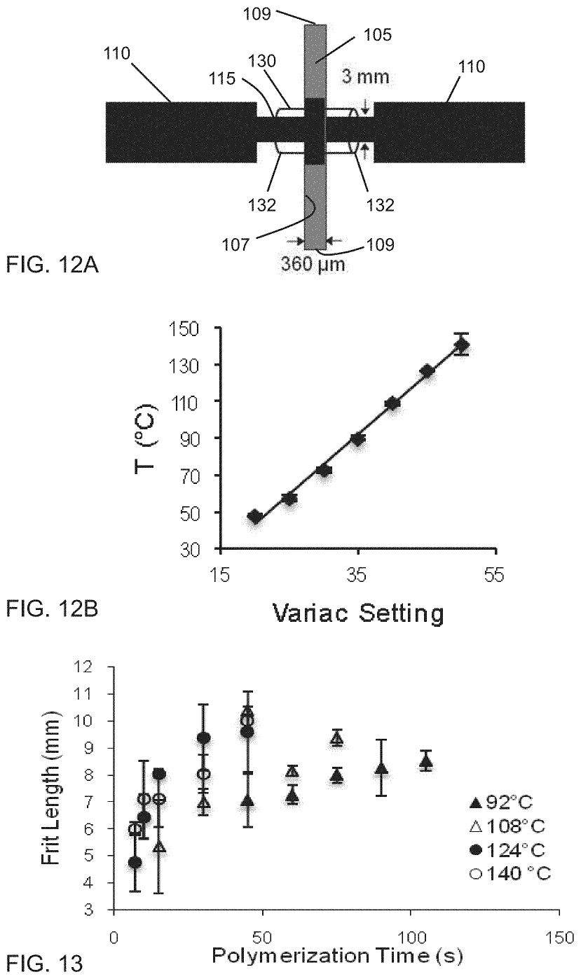

A new approach for thermal polymerization was developed to synthesize on-column polymer frits inside 100 μm i.d. capillaries. This methodology uses two soldering irons attached to VARIAC VAC transformers for temperature control, as shown in FIG. 12A. An aluminum alignment jig provided greater stability and positional reproducibility of soldering irons during polymerization, aiding in reproducible frit synthesis. The capillary can be easily positioned within the aluminum alignment jig allowing for the synthesis of a second retaining frit directly at the end of the packed bed. Referring to FIG. 12B, VARIAC VAC transformers allow for precise temperature control at the soldering iron tips. Also, the polyimide capillary sheathing is retained during frit synthesis, maintaining a stable capillary compared to UV polymerization. Overall, this method for thermal polymerization is rapid, precise, cost-effective, and maintains capillary stability.

In developing a new frit fabrication method, it was necessary to determine how various conditions affect frit polymerization. Referring to FIG. 13, several polymerization times and temperatures were used to determine the effect on frit length. Frit length can be controlled via a combination of polymerization temperature and time. As the polymerization time increased, the frit length increased. Frit sizes were shown to exceed the size of the soldering iron tips. Heat conduction that occurs as the soldering iron tips heat the monomer solution within the capillary, creating a diffuse heating zone for frit polymerization. These different conditions demonstrate the frit size is tunable to the experimental application, thus the desired frit length is readily obtained using appropriate polymerization time and temperature. This thermal initiation approach allows for rapid polymerization times<2 min compared to UV polymerization methods that necessitate a minimum of 1 h. To minimize band broadening in a separation, a relatively short frit with small deviation in frit size is desired. Frits<4 mm could be synthesized using smaller soldering iron tips.

In liquid chromatography, synthesized frits must withstand packing pressures and chromatographic backpressures without compressing or disintegrating. Pressure characterization was performed to compare the pressure stability of thermal and UV polymerized frits. The method uses a constant flow rate and monitored pressure for comparison of frit stability. The frit pressure stability was examined using Poiseuille's equation, which relates the linear velocity to the backpressure in the system (Equation 1):

μ₀=r²ΔP/8ηLₜ

where μ₀ is the linear velocity of the solvent, r the internal radius of the capillary tube, ΔP the applied pressure, Lₜ is the total length of the capillary and η the solvent viscosity. Based on Poiseuille's equation, the linear velocity and pressure within the system should exhibit a linear relationship.

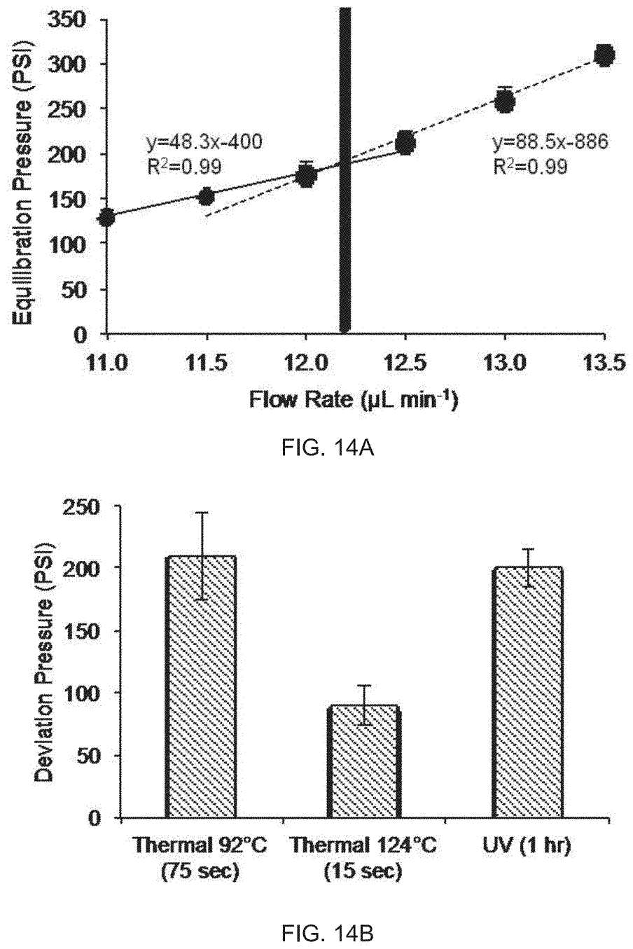

Any deviation from linearity would suggest morphological change, either compression or disintegration, in the frit. This deviation was used to determine the frit deviation pressure, as shown in FIG. 14A. The tested thermal synthesis conditions for pressure stability studies were 92° C. or 124° C. for 75 or 15 s, respectively. Both conditions produced precisely sized and relatively small 8 mm frits (frits of the same size are necessary for pressure comparison). Thermal initiated frits synthesized at 92° C. over 75s exhibited greater resistance to morphological change than frits synthesized at 124° C. over 15s by a statistically value of 125 psi. A longer polymerization time allows for greater conversion of the monomer and cross-linker, and thus greater cross-linking of linear polymer chains. Referring to FIG. 14B, thermal initiated frits synthesized at 92° C. over 75 s showed pressure stability of 200 psi over the 8 mm frit length, which is comparable to 1 h UV polymerized frits.

Frit stability throughout column packing is the ultimate measure of frit strength. The frit and packed bed can have the same porosity; therefore, the 200 psi pressure drop is equivalent to 7500 psi over a 30 cm packed bed. When packing a capillary, the distance over which the pressure in the system can drop continually increases as packing continues, lowering the amount of pressure per unit distance. Therefore, the thermal frits would remain robust during packing and chromatography. Due to greater frit pressure stability, the 92° C. for 75 s synthesis condition was chosen for use in packed capillaries.

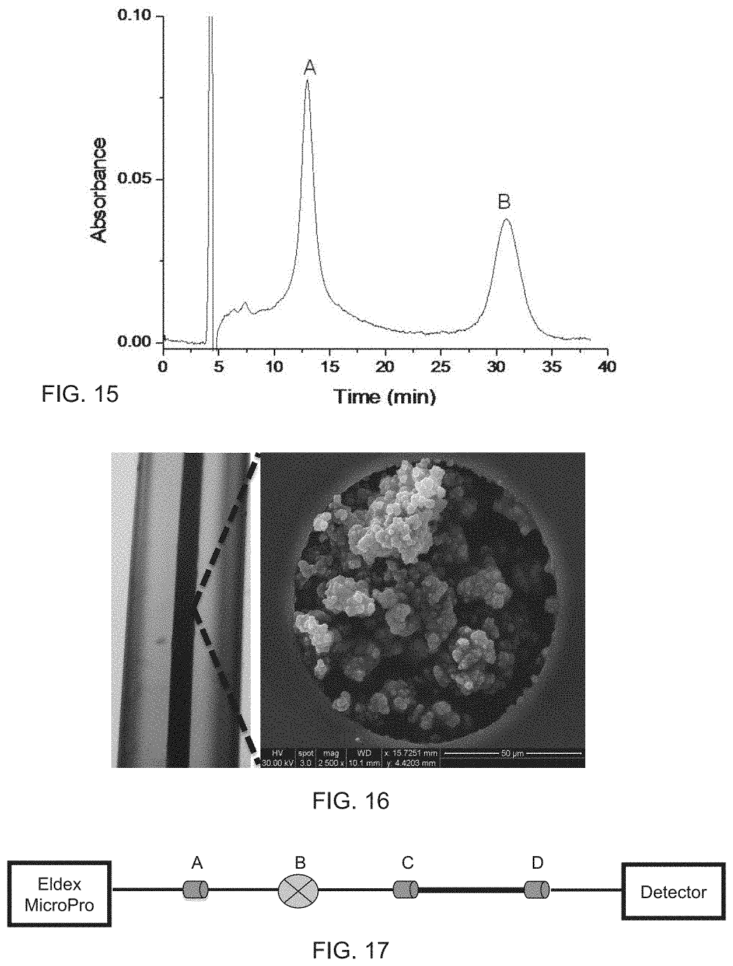

Packing a C₁₈-modified silica particle column with thermal initiated frits allowed for assessment of thermal frit stability and chromatographic performance. Thermal initiated frits synthesized at 92° C. for 75 s remained stable during packing and allowed for formation of a 17 cm packed bed of 5 μm diameter C₁₈-modified silica particles. This demonstrates thermal frit stability at 500 psi. The second thermal retaining frit was successfully synthesized and the packed capillary was used in cLC. As shown in FIG. 15, the thermal frit packed capillary allowed for the separation of FITC-labeled n-hexylamine and n-octylamine in 35 min at a flow rate of 2.0 μL min⁻¹.

Values of merit for reverse phase chromatography using a packed capillary with thermal frits are summarized in Table 1. This column allowed for resolution greater than baseline of the two analytes. Furthermore, the minimal error in retention time signifies the frits remain stable throughout multiple runs and allow for reproducible separations.

TABLE 1
Values of merit from cLC reverse phase separations
of FITC labeled aliphatic amines.

| Analyte | Retention Time (min) | Rₛ |
|---|---|---|
| FITC-Hexylamine (n = 3) | 12.95 ± 0.04 | 3.39 ± 0.09 |
| FITC-Octylamine (n = 3) | 30.86 ± 0.09 | |

The novel thermal polymerization method for preparing porous polymer frits has been developed and compared to an established UV polymerization method. The method allows for the synthesis of frits ranging from 4-10 mm depending on both the polymerization temperature and time, proving useful for tuning frit size to experimental application. Thermal frits exhibited sufficient porosity and robustness for pressure driven liquid chromatography. Additionally, the thermal initiated frits synthesized using the temperature-controlled polymerization allowed for reproducible separations of two aliphatic amines for use in a packed bed cLC.

This thermal polymerization method for polymer frits offers several advantages compared to existing methods. The soldering iron setup for thermal polymerization is much more affordable compared to expensive lamps needed for UV polymerization. The method allows for rapid polymerization times<2 min compared to ≥1 h for UV polymerization. Soldering irons allow for ease in placement of the second retaining frit with precise temperature control provided by VARIAC VAC transformers. The UV method necessitates removal of the capillary sheathing, thereby introducing fragility; whereas thermal polymerization allows for protection of the polyimide sheathing. Overall, this approach offers cost efficiency, fast polymerization times, simplicity, spatial precision and less fragile capillaries.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. The reference numbers recited in the below claims are solely for ease of examination of this patent application, and are exemplary, and are not intended in any way to limit the scope of the claims to the particular features having the corresponding reference numbers in the drawings. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A functionalized microparticle for selecting a ligand, comprising:
a. a microparticle core;
b. a plurality of lipid vesicles bound to a surface of the microparticle core, each vesicle comprising a lipid bilayer having a polymerized crosslinked structure; and
c. one or more target receptors embedded in the lipid bilayers of the vesicles, wherein the one or more target receptors are specific to the ligand.

2. The functionalized microparticle of claim 1, wherein the microparticle is a silica particle.

3. The functionalized microparticle of claim 1, wherein a diameter of the microparticle is between about 1 to 10 μm.

4. The functionalized microparticle of claim 1, wherein the vesicles have a diameter of about 100-600 nm.

5. The functionalized microparticle of claim 1, wherein the polymerized crosslinked structure is polymerized by thermal polymerization.

6. The functionalized microparticle of claim 1, wherein the lipid bilayer comprises polymerizable lipid monomers and functionalized lipid monomers.

7. The functionalized microparticle of claim 6, wherein the polymerizable lipid monomers are sorbyl- or dienoyl-containing lipid monomers.

8. The functionalized microparticle of claim 6, wherein the functionalized lipid monomers are amine-functionalized lipid monomers.

9. The functionalized microparticle of claim 8, wherein an amine functionality of the amine-functionalized lipid monomers is disposed outwardly and away from the vesicle.

10. The functionalized microparticle of claim 9, wherein the amine-functionalized lipid monomers comprise amino(polyethylene glycol) (NH₂-PEG).

11. The functionalized microparticle of claim 1, wherein the one or more target receptors are membrane protein receptors or lipid-derived receptors.

12. The functionalized microparticle of claim 1, wherein the lipid bilayer comprises a plurality of non-polymerizable lipid monomers and a plurality of polymerized, hydrophobic non-lipid monomers.

13. The functionalized microparticle of claim 12, wherein the non-lipid monomers are hydrophobic.

14. The functionalized microparticle of claim 12, wherein the lipid monomers are cell membrane fragments, phosphatidylcholine monomers, naturally occurring lipids, or synthetic lipids.

15. The functionalized microparticle of claim 1, wherein the surface of the microparticle is modified to provide a covalent attachment point for the lipid bilayer of the vesicles.

16. The functionalized microparticle of claim 1, wherein the surface of the microparticle is sulfonate-modified such that the surface comprises sulfonate molecules, wherein the sulfonate-modification provides a covalent attachment point for the lipid bilayer of the vesicles.

17. The functionalized microparticle of claim 1, wherein the polymerized crosslinked structure stabilizes the lipid bilayer.

18. An assay platform for selecting a ligand, said assay platform comprising a plurality of functionalized microparticles from claim 1, wherein the plurality of functionalized microparticles is mixed into a solution comprising the ligand, the ligand binds to a receptor of the one or more target receptors to form a ligand-bound assay platform, wherein the ligand-bound assay platform is removed from the solution, and detected via an analytical instrument, wherein when the ligand is detected, the ligand is identified by the receptor that is specific to the ligand.

19. A method for selecting a ligand using a plurality of functional microparticles from claim 1, wherein the method comprises mixing the plurality of functionalized microparticles with a solution comprising the ligand, wherein the ligand binds to a receptor of the one or more target receptors to form a ligand-bound receptor.

20. The method of claim 19 further comprising removing the ligand-bound receptor from the solution, and detecting the ligand using an analytical instrument.
